(19) United States
(12) Patent Application Publication
(10) Pub. No.: US 2026/0345902 A1
(43) Pub. Date: Nov. 5, 2026

(54) Method and System for Providing Non-invasive Control of Fluid Flow in Elastomeric Tubes

(71) Applicant: Byonyks Medical Devices, Inc., Itasca, IL (US)

(72) Inventors: Farrukh Usman, Itasca, IL (US); Michael Wollowitz, Itasca, IL (US); Ahmed Alam Ansari, Lahore (PK); Abdul Qadeer, Lahore (PK)

(21) Appl. No.: 19/117,993

(22) PCT Filed: Sep. 28, 2023
(86) PCT No.: PCT/US2023/033920

§ 371 Date: Apr. 3, 2025

Related U.S. Application Data
(60) Provisional application No. 63/412,570, filed on Oct. 3, 2022.

(51) Int. Cl. A61M 5/168

(52) U.S. Cl. CPC A61M 5/16881

(57) ABSTRACT

The present invention is a non-invasive flow control device that can control fluid flow in elastomer tubes in a non-invasive, normally closed, and antagonistic manner. Furthermore, it comprises a mechanical linkage and spring-based assembly of two plungers that are used to control fluid flow in two separate elastomer tubes. A single actuator is used to control the two plungers. The invention works primarily by pinching tubes through the plungers to restrict flow and relying on their elastomeric properties to allow flow when the plungers are sufficiently retracted. The primary mechanism of the invention is usable with different tube mounting mechanisms. The said invention may also be used in conjunction with a cassette where the plungers interact with an elastomeric membrane or diaphragm to obstruct or open a fluid pathway.

### CROSS-REFERENCE TO RELATED APPLICATIONS

This Patent Application claims priority to U.S. Provisional Patent Application No. 63/412,570, filed on Oct. 3, 2022. The disclosure of the prior Application is considered part of and is incorporated by reference into this Patent Application.

### FIELD OF INVENTION

The present disclosure is directed to a Non-invasive Flow Control in Elastomeric Tubes with several applications spanning the medical, food, and pharmaceutical industries, among others.

### BACKGROUND

Control of flow rate and start-stop operation of fluids is necessary for several applications spanning the medical, food, and pharmaceutical industries, among others. In many cases, the fluid (which may be gas or liquid) must remain isolated from the environment to prevent contamination either of the fluid by the environment or the surrounding environment by the fluid. In medical and laboratory practice, a disposable “set” may be used to transfer fluid from e.g., a sterile container to a patient. The set is pre-sterilized and disposed of after a single use, thereby minimizing the risk of contamination.

Any method to control or start-stop fluid flow where external components do not directly contact the fluid is termed non-invasive. In contrast, methods in which external components touch or otherwise influence fluid flow invasively are termed invasive methods. For example, a simple water faucet controls water flow invasively since valve components are in direct contact with water. Such an invasive method is common in various industrial applications. However, invasive methods are unacceptable in many applications such as the medical devices industry.

Specific applications may involve the flow of fluids in and out of patients' bodies through disposable and sterile sets, such as in dialysis. If invasive components are used in such applications, it is necessary for them to be sterile, and repeated usage would necessitate regular sterilization of said components. Otherwise, such invasive components would necessarily be parts of sterile disposable sets which are then thrown away after use, eliminating the risk of contamination but wasteful and expensive.

In certain applications, the fluid flow must be automated and is part of a controlled process. Therefore, the method, invasive or non-invasive, will necessitate the use of an actuator that limits or eliminates the manual operation of the fluid flow components.

One non-invasive fluid flow control approach is to place components outside the fluid path and interact with the fluid channel through the flexible periphery of the fluid path. One general implementation of this approach operates by pinching an elastomeric, fluid conveying tube against a rigid counter surface using a wedge-shaped profile (here referred to as a “plunger”). When the tube is pinched with sufficient force, the fluid channel is completely pressed close, fluid flow is restricted. When the pinching force on the tube is removed by retracting the plunger, the elastomer tube regains some or all of its original undeformed form, and fluid flow is restored partially or fully.

The present invention employs an actuated assembly that operates two plungers that can control fluid flow in elastomer tubes in a non-invasive, normally closed, and antagonistic manner.

The elimination of invasive contact of the valve elements from the fluid pathway decreases the exposure of the sterile fluid pathway to external elements and reduces the risk of contamination and infection in medical and/or pharmaceutical inventions.

The invention can also be easily automated and interfaced with a control system, allowing the invention to be a part of an automated production process or a computer-controlled machine.

Here is a brief analysis of the prior art cases to ensure and emphasize the novelty of the present invention. 1. Fluid flow control valve and transfer set, U.S. Pat. No. 4,821,996, Baxter the invention in this prior art claims the methods and devices used in connection with carrying out peritoneal dialysis. More particularly, the invention pertains to a valve system, fluid transfer set, and method that facilitates executing the steps of a drain and fill cycle associated with continuous ambulatory peritoneal dialysis. However, the present invention is related to general applications of fluid flow control through elastomeric tubes including those of Continuous Ambulatory Peritoneal Dialysis (CAPD) and Automated Peritoneal Dialysis (APD). In addition, the present invention is controlled through automated means (actuator-driven) whereas the prior art would manually control the fluid flow. Furthermore, the present invention is fail-safe in case of power failure, whereas the prior art requires manual intervention to start, stop or control fluid flow. The present invention is intended to be used with sets of single continuous elastomer tubes than the prior art that can be used with three tubes that connect to the invention's outlets. The present invention is a completely non-invasive fluid flow control method that is non-disposable, but the prior art is invasive in its working which necessitates that this invention is disposed of after use in sterile applications. 2. Flow control device for peritoneal dialysis, US 2009/0143723 A, Baxter A peritoneal dialysis flow control device in one embodiment includes (i) the first cap including a first medical fluid line connection and a second medical fluid line connection; (ii) a gasket mated with the first cap where the gasket defines a first aperture in fluid communication with a first port and a second aperture in fluid communication with a second port; and (iii) a second cap including a third medical fluid line connection where the second cap is sealed rotatably to the gasket.

The above invention serves the same purpose as ours which is “controlling fluid”. The difference is that they are controlling fluid flow invasively whereas the present invention is non-invasive. For sterile applications, there would be a necessity to use a new copy of this invention whereas the present invention is non-disposable even for sterile applications. This invention makes use of rotary components to control fluid flow whereas the present invention makes use of linear plungers which interact directly with elastomer tubes without contact with the fluid.

Lastly, the present invention can be used in general fluid flow applications including but not limited to peritoneal dialysis whereas the prior art is specifically intended for peritoneal dialysis.

### SUMMARY

A first example includes a system for a non-invasive Flow Control in Elastomeric Tubes, as shown in FIG. 1, comprising a spring-based assembly 18 of two plungers 15, 22, an actuator motor 11, and Elastomer tubes 37. The motor is available in various possible forms that are back driven and can maintain a position when powered. The plungers are spring-loaded in a normally closed configuration and are partially actuated in the opening direction through a mechanical linkage. The plunger is antagonistic in action, such that only one plunger can open at once. However, the spring action and the design of the mechanical linkage allow both plungers to be in the closed state simultaneously. The closing force exerted by the plungers deforms the elastomer tubes such that the path of the fluid is blocked in the closed state. When the plungers are pulled back by the action of the actuator and the linkage, the path is restored.

A second example includes the method of example 1 in which the mechanical linkage is a four-bar linkage.

A third example includes the method of example 1 in which the mechanical linkage is allowed to operate at or near the toggle position such that the spring force on the actuator is near zero. This allows low power operation of the actuator which may be operated at lower power settings or the power to the actuator may be completely cut off.

A fourth example includes the method of example 1, in which the mechanism is restricted to operate near the toggle point such that the invention is fail-safe by design. This means that when the actuator is powered and opening one of the plungers against the spring, the spring force exerts sufficient force or torque on the actuator to allow the plunger to return to the closed state in case of power failure to the actuator, A fifth example includes the method of examples 1, 2, 3 and 4, where the plungers are wedged-shaped profiles, without any emphasis on specific profile designs, which push against elastomer tubes perpendicularly to the tube axis with sufficient force to close the walls of the tubes and restrict fluid flow. The plunger 22 may be integral to the plunger shaft 15 or may be separately mounted on the plunger shaft by means such as interference fit, mechanical fastening, or adhesives, among other methods.

A sixth example includes the methods of the previous examples, where the invention uses a closed loop control system and a sensor to track the position and open-closed state of the system.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 is an elevation view of the present invention.

FIGS. 2A and 2B show an isometric view of the invention with four-bar linkage, two plungers in the closed position, springs, and a driving motor.

FIGS. 3A and 3B a different isometric, plan, and elevation views of the invention with four-bar linkage and springs with the top plunger in the open position.

FIG. 4 shows the front elevation view of the invention with a four-bar linkage that operates away from the toggle position of the linkage.

FIGS. 5A and 5B show the front elevation views of the invention with a four-bar linkage that operates near or at the toggle position of the linkage.

FIG. 6A-6H shows various front elevation and isometric views of the invention, showing one possible method of mounting the elastomeric tubes on a pivoting mounting assembly. One sub-figure shows the sliding assembly being actuated by an actuator.

FIG. 7A-7H show various front elevation and isometric views of the invention, showing one possible method of mounting the elastomeric tubes on a static mounting assembly.

FIGS. 8A and 8B show the front elevation, sectional, and isometric views of the invention in use with a cassette. These Figures show the fluid pathways in detail.

FIGS. 9A and 9B are a front elevation and isometric view of a symmetric rocker.

### DETAILED DESCRIPTION

The foregoing disclosure provides illustration and description but is not intended to be exhaustive or to limit the implementations to the precise form disclosed. Modifications may be made in light of the above disclosure or may be acquired from the practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limited of the implementations. Thus, the operation and behavior of the systems and/or methods are described herein without reference to specific software code—it is understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

The present disclosure is directed to a Non-invasive Flow Control in Elastomeric Tubes with several applications spanning the medical, food, and pharmaceutical industries, among others. The components included in the system and their functions are delineated below:

Unless defined otherwise, the terminologies and terms used herein have the same meaning as commonly understood by one having ordinary skill in the field to which this invention belongs. It is understood that the general terms are not interpreted with a meaning which is not defined herein. Here is a detailed description of the present invention by referring to the given figures:

### Description of the Components

The Rotary Motor 11 is an electrical actuator in some of the embodiments.

The Motor 11 is mounted on a Plate 1 and this plate provides a means for mounting the actuator.

A crankshaft 2 is a metallic or otherwise shaft that mounts the Crank 3.

The Crank 3 is the driving link in the mechanical linkage. It may be mounted directly onto the actuator or through a linkage or gear transmission.

The Crank Pin 4 is the pin on the crank 3 that interacts with the slot in the rocker in one embodiment.

The rocker 9 is a part of the mechanical linkage in one embodiment that pulls the plunger shaft 15 against the springs through the plunger pin 16.

Rocker Shaft 10 is a metallic or otherwise shaft that provides a pivot for the rocker.

The Plunger Plate 12 simply provides a mechanical structure for the linear bearings for the plunger shaft 15.

The Plunger Shaft 15 forms a part of the plunger 22 in some embodiments.

The Plunger Pin 16 is a pin on the plunger that interacts with the rocker.

Spring 18 is a helical compression spring.

A plunger 22 is a wedge-shaped profile that interacts with the elastomeric tubes.

The retaining Ring 27 is a common mechanical fastening element.

Mounting Assembly 34, 35, 36, 38 is a structure for mounting the elastomeric tubes in some of the embodiments.

The lead screw 35 is a common mechanical transmission element.

The organizer tray/slider 36 is a part of the mechanical structure of the mounting assembly 34.

An Elastomeric Tube or elastomer tube 37 is a flexible tube intended for fluid flow.

Hinge/Pivot 38 is simply a pivot for a rotating body.

Mounting Assembly Actuator 40 is an actuator in the mounting assembly in some of the embodiments.

Fluid Inlet Path 41 is a path for the fluid to flow.

Fluid Outlet Path 42 is a path for the fluid to flow.

Countersurface Washer 43 is an elastomeric washer that deforms to form a sealable periphery.

The diaphragm 44 is a flexible membrane.

A cassette 45 is a body in some of the embodiments with several fluid pathways that can be opened or closed as needed.

Virtual Crank Link or Crank Axis 50 is the line running along the Crank 3 through the center of the crank pin 4 and the crankshaft axis.

The virtual Rocker and Slider Link (Rocker Axis) 51 is the line joining the center of the plunger pin 16 and the crank pin 4 on the rocker 9 through the rocker.

The translating Axis for the Top Plunger Shaft 60 is the center axis of the top plunger shaft 15.

The translating Axis for the Bottom Plunger Shaft 61 is the center axis of the bottom plunger shaft 15.

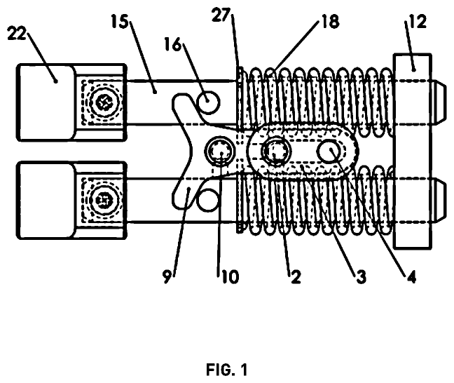

FIG. 1 shows the front elevation view of a spring-based assembly 18 of two plungers 15, 22 that are used to control fluid flow in two separate elastomer tubes through a four-bar linkage.

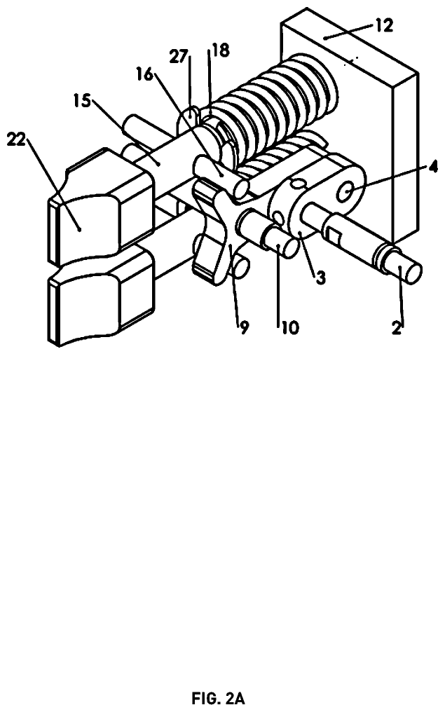

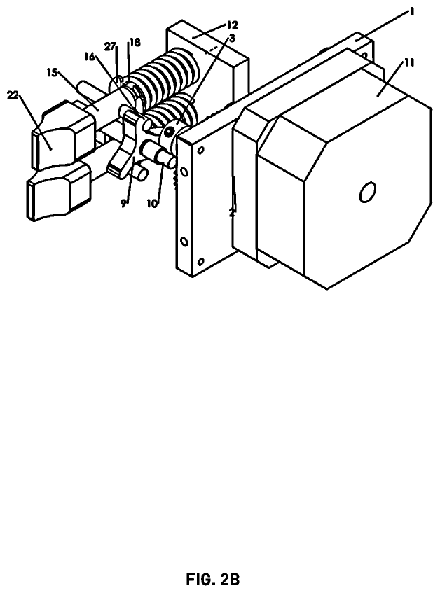

FIG. 2A illustrates the isometric view of the identical apparatus as FIG. 1, which shows a mechanical linkage where the rocker 9 has two extended arms at specific angles about the rocker mid-plane. The crank 3 is driven by a rotary motor (explained below) and in turn, moves the pin 4 inside the rocker slot thereby rotating the rocker in a certain direction. The rocker's arm pushes against a pin 16 on the plunger to open it against the spring 18.

FIG. 2B illustrates the isometric view of the identical apparatus as FIGS. 1 and 2A, which shows the rotary motor 11 connected to the crankshaft 2 which drives the crank 3.

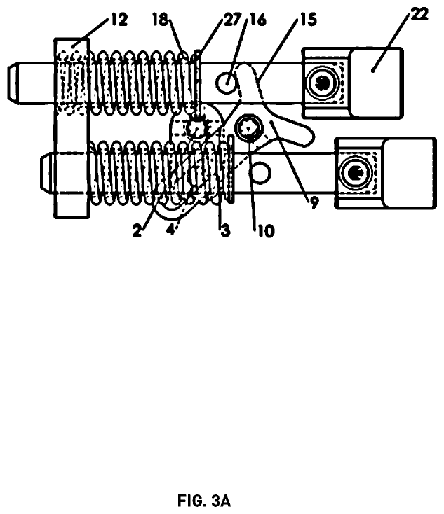

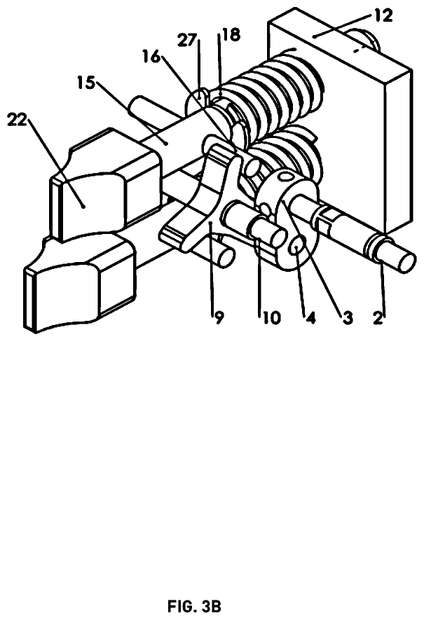

FIG. 3A shows the front elevation view of an identical apparatus as FIG. 1, with the top plunger 15,22 in the open state. The bottom plunger remains unaffected and in a closed state.

FIG. 3B shows the isometric view of an identical apparatus as FIG. 2A, with the top plunger 15,22 in the open state.

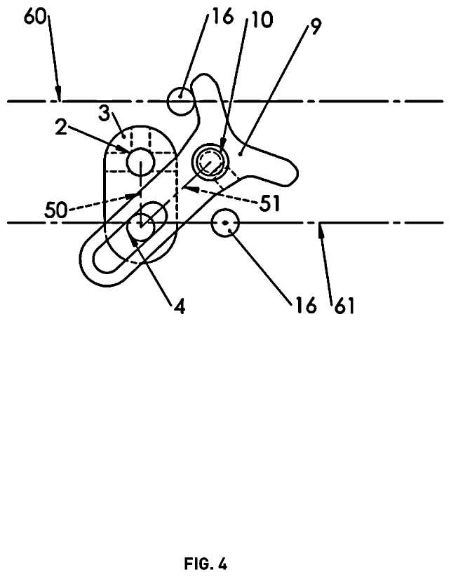

FIG. 4 shows the front elevation view of the four-bar linkage. For the invention to be fail-safe, the spring force should exert sufficient torque through the mechanism at the crank 3 to overcome the motor cogging torque. This can be set by changing the link dimensions of the four-bar mechanism and/or changing the spring stiffness. In such an embodiment, the four-bar mechanism must operate away from the toggle position This may be achieved by mechanically limiting the rotational travel of the crank or the rocker. The figure shows the non-perpendicular angle between the crank axis 50 and the rocker axis 51 which results in a non-zero torque at the crank 3 due to spring force.

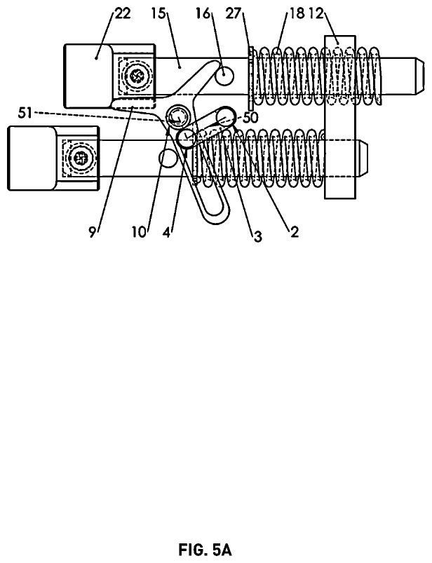

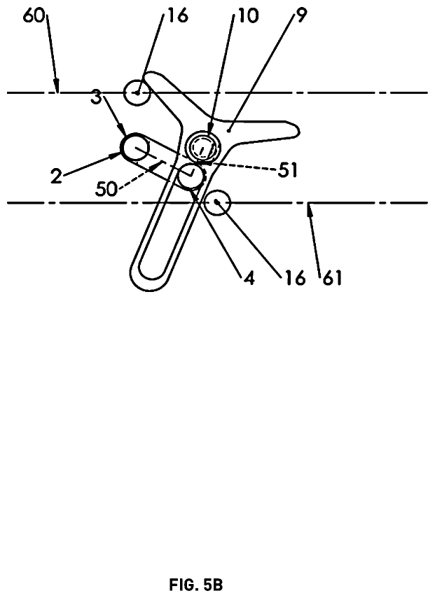

FIG. 5A shows the front elevation view of the invention with modified geometries of the crank 3 and the rocker 9 which allow the linkage to be positioned at a perpendicular or near-perpendicular angle between the crank axis 50 and the rocker axis 51 At this toggle position, the force of the springs will result in a zero or near-zero torque at the crank 3. The rotary motor may be switched off or operated at reduced torque at this angle to save power and reduce heat generation. In such an embodiment, the invention no longer remains fail-safe upon power failure. Such an embodiment may be useful in applications when fail-safety is not required, and improved energy efficiency is desirable.

FIG. 5B shows the elevation view of the embodiment with a greater focus on the four-bar linkage.

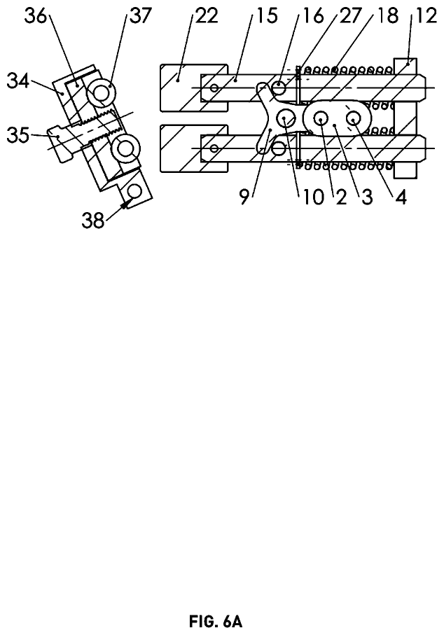

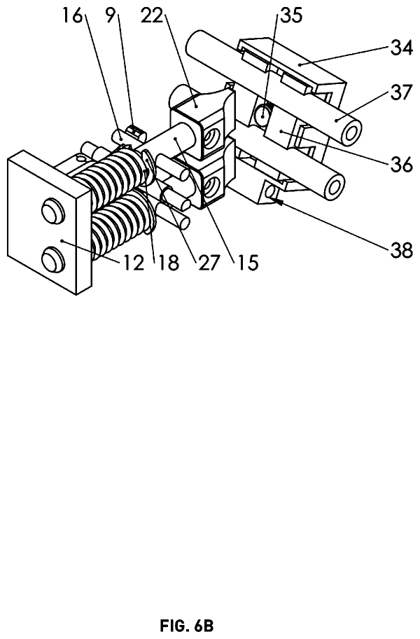

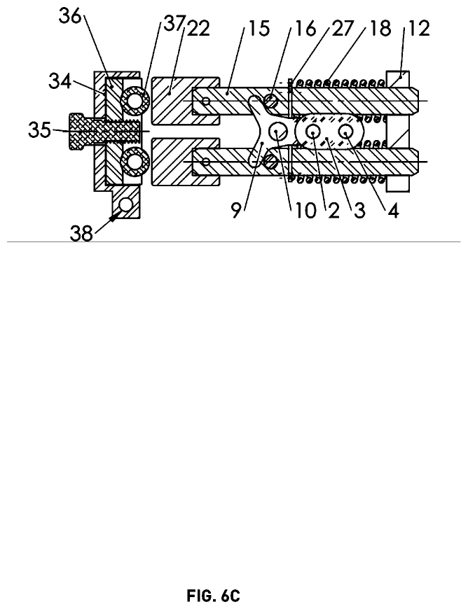

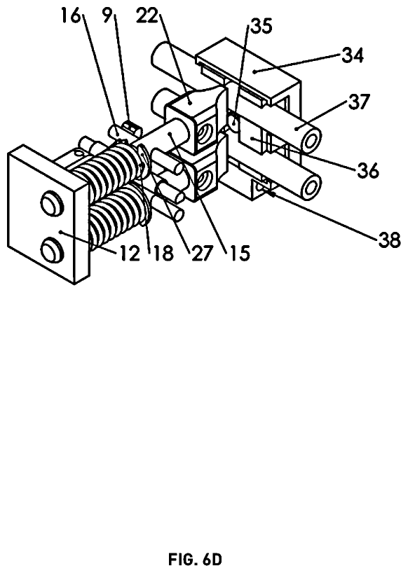

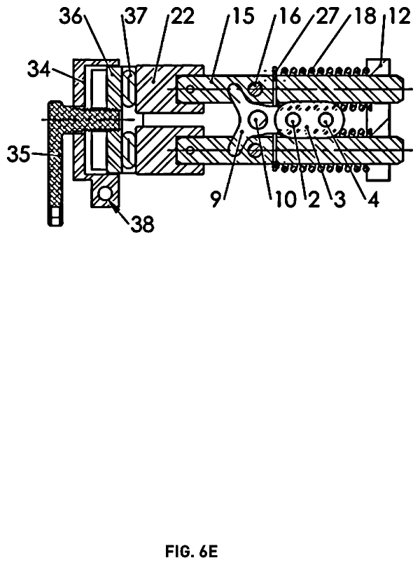

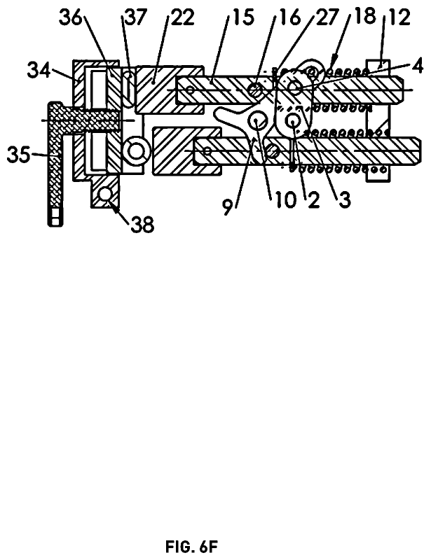

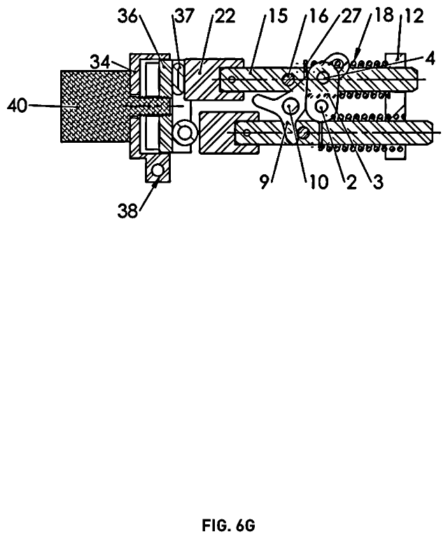

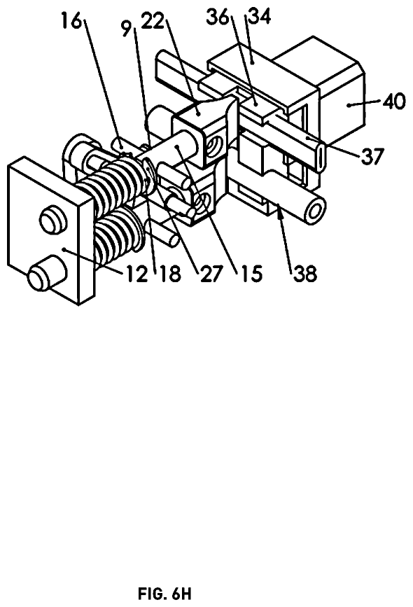

FIG. 6A shows the invention consisting of a mounting assembly 34, 35, 36, 38 that allows the user to easily place the tubes 37 in front of the plunging mechanism. In this embodiment, the tubes can be organized using an organizer tray 36 which can then be placed inside the mounting assembly. The sliding mechanism 35, 36 in the mounting assembly is a leadscrew & threaded hole linear-motion combination that can then gradually load the tubes against the closed plungers. In such a case, both tubes 37 will be in a closed state when loaded.

FIG. 6B to FIG. 6F shows different elevation and isometric views of the apparatus.

FIG. 6G shows the elevation view of another embodiment similar to the one shown in FIG. 6A. In this embodiment, the sliding mechanism can be actuated using a motor 40, hydraulic actuator, or pneumatic actuator, amongst others.

FIG. 6H shows the isometric view of the embodiment presented in FIG. 6G.

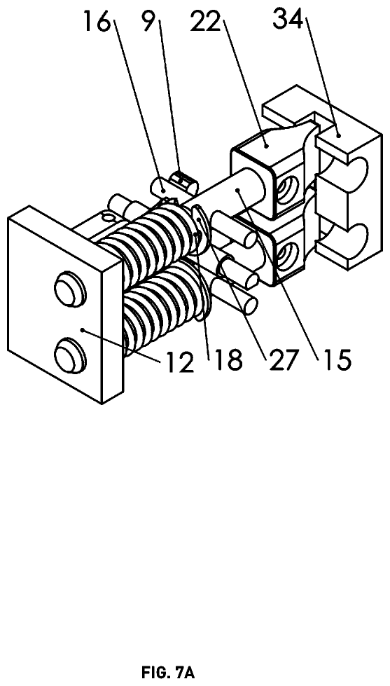

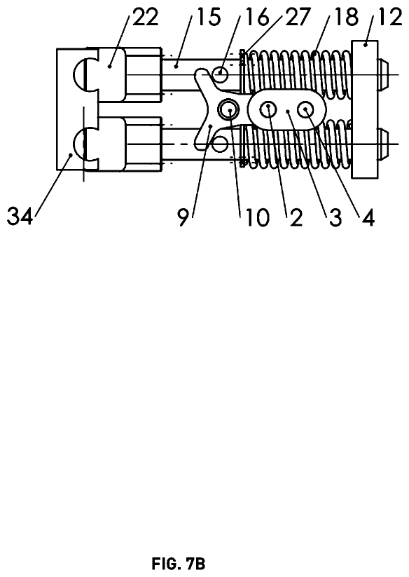

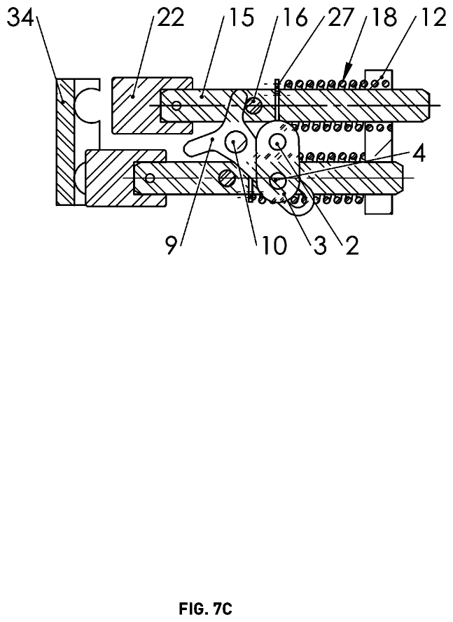

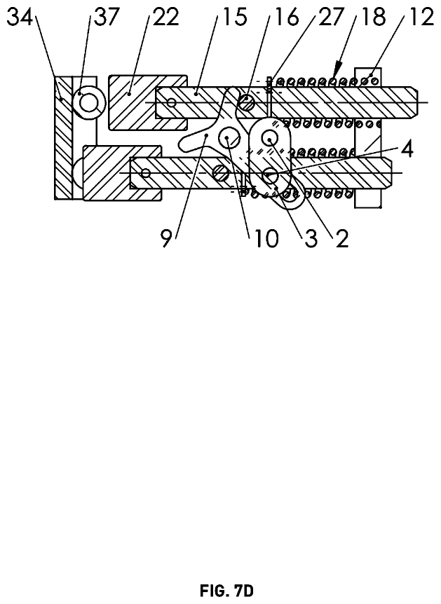

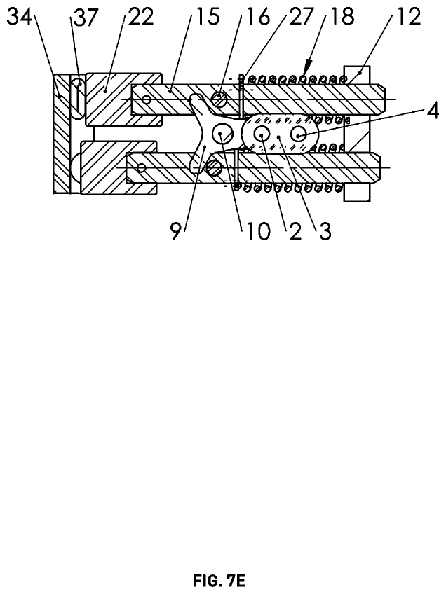

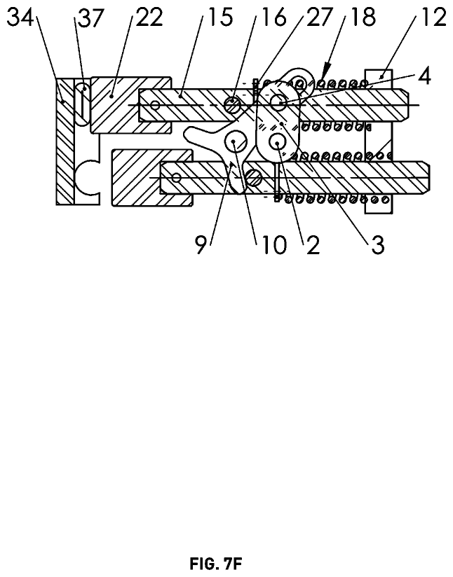

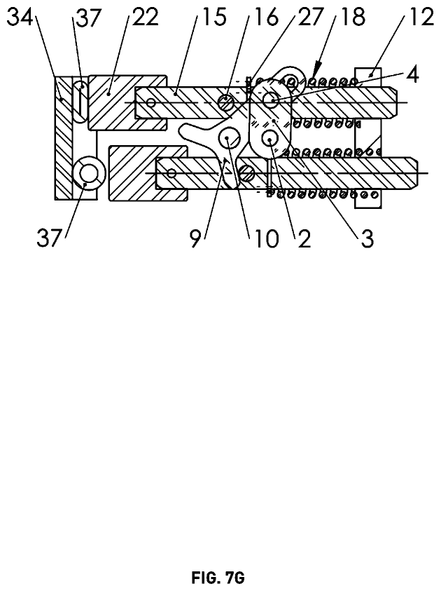

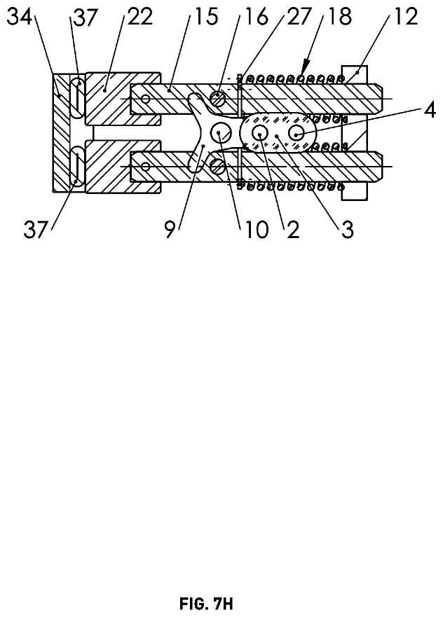

FIG. 7A shows the isometric view of an embodiment consisting of a simple rigid counter surface 34 that may be used with the plunging mechanism. In such an embodiment, one of the plungers 22 may be retracted to allow the initial placement of the first tube easily. The second plunger may then be retracted to allow initial placement of the second tube easily.

FIG. 7B shows the front elevation view of the embodiment presented in FIG. 7A.

FIG. 7C shows the front elevation view of the embodiment presented in FIG. 7A, with the top plunger in the open state.

FIG. 7D shows the front elevation view of the embodiment presented in FIG. 7A, with the top plunger in the open state. The tube 37 is then placed when the top plunger is retracted.

FIG. 7E shows the front elevation view of the embodiment presented in FIG. 7A, with the top plunger in the closed state and pushing against the top tube 37.

FIG. 7F shows the front elevation view of the embodiment presented in FIG. 7A, with the bottom plunger in the open state.

FIG. 7G shows the front elevation view of the embodiment presented in FIG. 7A, with the bottom plunger in the open state. The tube 37 is then placed when the bottom plunger is retracted.

FIG. 7H shows the front elevation view of the embodiment presented in FIG. 7A, with the bottom plunger in the closed state and pushing against the bottom tube 37.

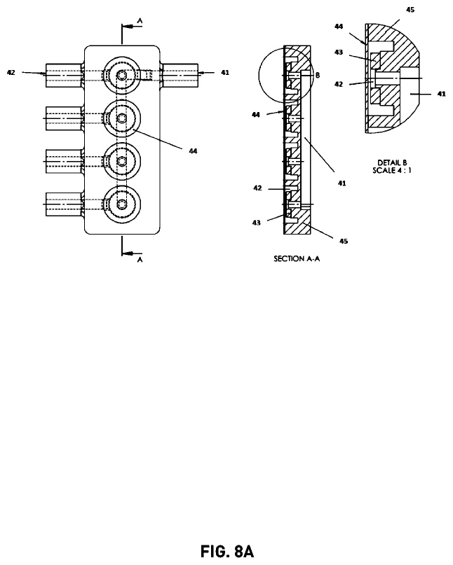

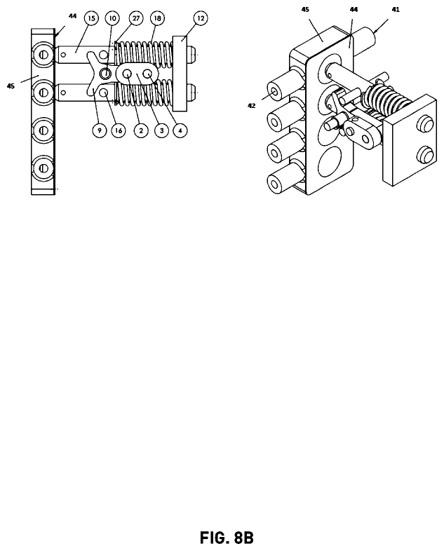

FIG. 8A shows the front elevation and side sectional views of the cassette 45 and internal fluid pathways. The fluid inlet 41 is connected to the fluid outlet 42. This connection can be obstructed by pushing the diaphragm 44 against the countersurface washer 43.

FIG. 8B shows the side elevation and isometric views of the cassette 45 with the plunging mechanism. The plunger 15 pushes against the diaphragm 44 to obstruct or clear the pathway between the fluid inlet 41 and the fluid outlet 42 as shown in FIG. 8A.

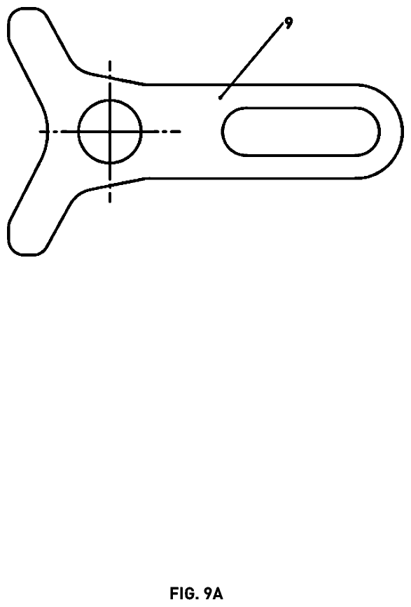

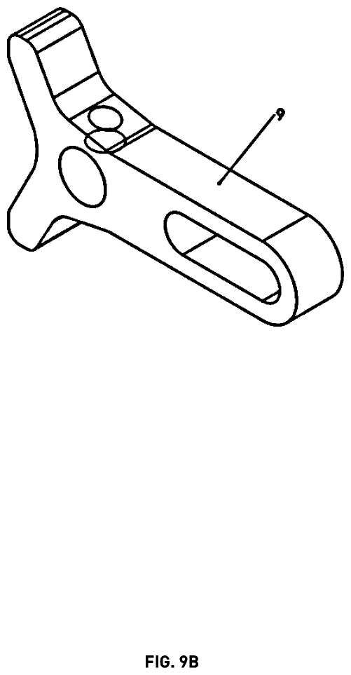

FIG. 9A shows the front elevation view of a symmetric rocker.

FIG. 9B shows the isometric view of a symmetric rocker.

### Embodiments in Detail The invention employs a mechanical linkage. All embodiments covered in this document employ four-bar linkages, but other linkages may be used with identical results.

The rocker 9 has two extended arms at specific angles about the rocker mid-plane. The rocker is depicted separately in FIGS. 9A and 9B. These angles may be symmetric about the rocker mid-plane or may differ, depending on specific implementations. The crank 3 is driven by a rotary motor (explained below) and in turn, moves the pin 4 inside the rocker slot thereby rotating the rocker in a certain direction. The rocker arm then pushes against a pin 16 on the plunger. The plunger then moves linearly in a set of bushes or linear bearings (the supporting bearing arrangement is not depicted herein). In the arrangement shown in FIG. 3A, the rotation of the crank in a certain direction will retract or open the top plunger. The bottom plunger is not influenced in this case and will remain in the closed position. When the crank returns to its zero (horizontal) position, the plungers will both be in a closed state.

In one embodiment, shown in FIG. 4, the invention is fail-safe. In the event of loss of motor torque due to power failure or other failures, the springs 18 push the plunger back against tube 37 to the closed state. If one of the plungers was in the open state when the power failure occurred, the spring will push against the plunger which will then push against the rocker's arm through the plunger pin 16. The rocker arm will resultantly restore the crank 3 to the normally closed/neutral position against the motor cogging torque. For the invention to be fail-safe, the spring force should exert sufficient torque through the mechanism at the crank to overcome the motor-cogging torque. This can be set by changing the link dimensions of the four-bar mechanism and/or changing the spring stiffness. The four-bar mechanism must operate away from the toggle position in such an embodiment. This may be achieved by mechanically limiting the rotational travel of the crank or the rocker.

In another embodiment, shown in FIGS. 5A and 5B, the invention is self-locking. If the crank axis 50 is allowed to move to a position that is perpendicular to the rocker axis 51, the four-bar mechanism will be at the toggle position, such that the output force of the springs will result in zero torque at the crank 3. The motor may be switched off or operated at reduced torque at this angle to save power and reduce heat generation. In such an embodiment, the invention no longer remains fail-safe upon power failure. Such an embodiment may be useful in applications when fail-safety is not required and improved energy efficiency is desirable.

In all embodiments of the said invention, a motor (as shown in FIGS. 2B, 11) is used to drive the crank 3. The motor is not depicted in other illustrations for clarity; the mounting means for the motor are also not illustrated. The motor may be an AC or DC motor, including a servo motor or stepper motor. The motor may drive the crank directly or through internal or external gearing. There is no preference for motor type although certain applications may prefer particular motor types. For the invention to be fail-safe, the torque at the motor shaft, in case of power failure, must overcome any residual torque of the motor or associated drive components so that the crank is restored to its neutral position, as described before. This characteristic of the motor is often referred to as “back-drive-ability” and is mostly a subjective parameter. The residual torque is also referred to as “open-circuit torque”, “cogging torque” or “detent torque” in the case of stepper motors, and is the torque required to rotate the motor shaft when it is unpowered. As noted earlier, embodiments that are not fail-safe, may or may not involve back-driveable motors since the four-bar toggle positioning makes the mechanism non-fail-safe (self-locking) regardless of the degree of back-drive-ability of the motor.

The motor itself may be run in open-loop or closed-loop positional control. A rotational position sensor, such as a non-contact hall effect sensor, may be used to obtain position feedback from the angular position of the motor shaft, crankshaft 2, or rocker shaft 10. This feedback may then be used for motor positional control as well as to obtain information about which valve is opened or closed. An absolute position sensor is preferred to set up a consistent zero reference position at the neutral position of the crank even when power to the invention is turned off. As an alternative, limit switches may be used to detect the linear limits of the two plunger shafts 15 to control the rotational limits of the motor. These limit switches may be of the contact or non-contact type (such as photo interrupters) without any preference, although certain applications may prefer particular types.

The invention may be used in two broad ways: 1) for start-stop control of fluid flow, and 2) to control the rate of fluid flow in an open-loop or closed-loop system.

The invention is also partially actuator-independent such that the plungers can be moved in the open direction without any actuator influence. This invention allows tubes of different sizes (ID, OD, and wall thickness) and hardness to be utilized. The springs 18 can be sized to control the closing force on the tube in the closed position. The linear distance required to allow fluid flow can be controlled by designing the link lengths accordingly. The plunger's total stroke depends upon the four-bar mechanism's linkage lengths. By applying trigonometric relationships, a relationship can be developed that relates the link lengths, the driving crank angle, and the stroke. Link lengths also dictate the torque required at the crank for compressing the spring.

In an embodiment, as shown in FIG. 6A, the invention consists of a mounting assembly 34, 35, 36, 38 that allows the user to easily place tubes 37 in front of the plunging mechanism. In this embodiment, the tubes can be organized using an organizer tray 36 which can then be placed inside the mounting assembly. The sliding mechanism 35, 36 in the mounting assembly is a lead screw & threaded hole linear-motion combination that can then gradually load the tubes against the closed plungers. In such a case, both tubes 37 will be in a closed state when loaded. In another embodiment, the sliding mechanism can be actuated using a motor, hydraulic actuator, or pneumatic actuator, amongst others (40, FIGS. 6C, 6D).

In another embodiment shown in FIG. 7A, a simple rigid counter surface 34 may be used with the plunging mechanism. In such an embodiment, one of the plungers 22 may be retracted to allow the initial placement of the first tube easily. The second plunger may then be retracted to allow initial placement of the second tube easily.

In another embodiment, the invention may be used in conjunction with a “cassette” (45, FIGS. 8A, 8B) consisting of an elastomeric sheet or diaphragm 44. The cassette is supported rigidly by a specific arrangement. Such a diaphragm may be pushed against a countersurface washer 43 to open or block a 3-dimensional fluid path to allow or restrict fluid flow respectively. In FIG. 8A, the fluid inlet path 41 is normally open to the fluid outlet 42 when the diaphragm is in its relaxed state. When the diaphragm 44 is pushed inwards (towards 43) by the plungers 15 in the normally closed configuration described in earlier embodiments, the fluid pathway is blocked, and flow is restricted. When one of the plungers is retracted, the diaphragm is restored to its relaxed position, and/or positive pressure from the fluid inlet forces the fluid pathway from 41 to 42 to open, hence, fluid flow is restored. The “cassette” may be of several different designs depending on required fluid pathways. For example, in FIGS. 7A, 7B, all four outlets are connected to a common inlet, but this may differ in different applications. The number of inlets and outlets may also differ.

The design elements of the invention also permit one valve to be fail-safe by limiting the driving angle on that side, while the other valve is allowed to go into a self-locking state. Such a device may be useful in applications using two elastomeric tubes in which flow in one tube needs to be fail-safe, without any restrictions on fluid flow in the other tube. In such an embodiment, the driving angle will be asymmetrical and will allow zero or reduced energy expenditure when keeping the non-fail-safe valve open.

All embodiments are very useful in applications where antagonistic fluid flow in a pair of fluid pathways is required, i.e., when fluid flow is required in one out of two fluid pathways only and the other tube must necessarily restrict fluid flow.

In all embodiments, the invention can be multiplied to form plunging assemblies in multiples of 2, i.e., 2, 4, 6, 8, and so on. However, the working of the individual plunging assemblies will remain the same. Such an embodiment is useful in applications where flow in multiple tubes needs to be controlled such as automated peritoneal dialysis machines or pharmaceutical dispensing machines, amongst others.

The present invention pertains several benefits as it provides a ready solution for applications where antagonistic fluid flow action is required in two elastomeric tubes. It also allows different configurations to be used with each other to form plunging assemblies in multiples of two. A single motor controlling all the embodiments makes the control easier and more cost-effective. Furthermore, it allows features from several different embodiments to be merged into a single embodiment, e.g., an embodiment may contain features from both fail-safe and self-locking embodiments.

These, as well as other aspects, advantages, and alternatives, will become apparent to those of ordinary skill in the art by reading the following detailed description, with reference where appropriate to the accompanying drawings. Further, it should be understood that this summary and other descriptions and figures provided herein are intended to illustrate the invention by way of example only and, as such, that numerous variations are possible.

1. A mechanism comprising:
(a) rotating and sliding links;
(b) a motor with various possible forms that are back-drivable and can maintain a position when powered;
(c) a pair of spring-loaded plungers in a normally closed configuration, partially actuated, forming a four-bar independent linkage, and being antagonistic in action, all controlled by a single motor; and
(d) a rotary encoder with various possible forms that provide feedback on motor or linkage positions.

2. The mechanism of claim 1, further comprising a crank, a rocker, and a sliding link located between the crank and the rocker.

3. The mechanism of claim 1, wherein the mechanism is configured to prevent entering a toggle position and is configured to ensure that a valve remains normally closed in case of power failure to the motor.

4. The mechanism of claim 1, wherein the mechanism is designed to allow entering a toggle position capable of keeping a valve open without motor power.

5. The mechanism of claim 1, wherein the motor is electric.

6-7. (canceled)

8. The mechanism of claim 1, wherein the rotary encoder is configured to provide feedback as position data to a control system.

9. The mechanism of claim 1, further comprising a control system configured to adjust operation of the motor based on feedback from the rotary encoder to control fluid flow.

10. The mechanism of claim 1, wherein the pair of spring-loaded plungers are configured to act as redundant safety mechanisms configured to ensure proper valve closure and are independently adjustable to fine-tune valve control.

11. The mechanism of claim 1, further comprising a power backup system configured to ensure continued operation in case of power failure.

12. (canceled)

13. The mechanism of claim 1, further comprising a user interface for control and adjustment of valve settings.

14. The mechanism of claim 1, wherein the rotary encoder is equipped with position sensing technology configured to provide feedback to control fluid flow.

15. The mechanism of claim 1, wherein the mechanism is adapted for use in industrial processes, chemical plants, or water treatment facilities.

16-23. (canceled)

24. A method of operating the mechanism of claim 1, the method comprising preventing the mechanism from entering a toggle position in response to a power failure of the motor.

25. The method of claim 24, further comprising maintaining a valve in a closed position in response to the power failure.

26. The method of claim 24, further comprising allowing the mechanism to enter the toggle position after power is reinstated to the motor.

27. The method of claim 24, further comprising generating feedback in as position data to a control system.

28. The method of claim 27, further comprising operating the motor based on the feedback.

29. The method of claim 24, further comprising tuning valve control via the spring-loaded plungers.

30. The method of claim 24, further comprising providing a user interface for control and adjustment of valve settings.
